(19) United States
(12) Patent Application Publication
(10) Pub. No.: US 2003/0180350 A1
(43) Pub. Date: Sep. 25, 2003

(54) Combination compositions

(76) Inventors: Majid Hameed Abdul Razzak, Dunedlin (NZ); Suchat Watnasirichaikul, Dunedin (NZ); Ian George Tucker, Dunedin (NZ)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/332,749

(22) PCT Filed: Jul. 13, 2001
(86) PCT No.: PCT/NZ01/00140

(30) Foreign Application Priority Data
Jul. 13, 2000 (AU) PQ8757

(51) Int. Cl. A61K 39/00; A61K 31/7048; A61K 31/714; A61K 9/127

(52) U.S. Cl. 424/450; 514/28; 514/29; 424/185.1; 514/52

(57) ABSTRACT

The invention relates to a veterinary composition and in particular one that enables the incorporation of both hydrophilic and lipophilic drugs. In a first aspect the invention relates to a stable composition including at least one hydrophilic active and medium chain mono- and di-glycerides. Preferably the composition additionally includes at least one lipophilic active. Preferably the composition additionally includes at least one of a surfactant, solvent and a carrier. Preferably the hydrophilic is selected from the group including clorsulon, closantel, vaccine solution, sodium selenate, vitamin B12, and other hydrophilic anthelmintics and mineral salts. More preferably the lipophilic active is selected from the group including macrocyclic lactones, including avermectins and milbemycins.

### FIELD OF THE INVENTION

The invention relates to a veterinary composition and in particular one that enables the incorporation of both hydrophilic and lipophilic drugs.

### BACKGROUND

Generally drugs, dietary supplements, vaccines and the like (hereinafter “actives”) can be divided according to their solubility characteristics to form two broad classes, hydrophilic and lipophilic actives.

Hydrophilic actives are generally solubilized in water-based formulations. Lipophilic actives generally solubilize in oil-based formulations.

The terms “hydrophilic” and “lipophilic” refer to relative affinities for, and compatibility with, water versus typical oily/organic solvents. A simple test is to physically shake a sample of unknown material with a mixture of both water and a water immiscible organic solvent such as octanol, until equilibrium is attained, and allow the liquid phases to separate. A substance found predominantly in the water phase would be judged to be hydrophilic, while conversely a material found predominantly in the octanol phase would be considered lipophilic. The high lipophilicity of the drug is indicated by the value of the partition coefficient P in the system octanol/water. A hydrophilic moiety that increases the lipophilicity of the drug and may increase its octanol/water partition coefficient.

The term “microemulsion” refers to a thermodynamically stable dispersion of one liquid phase into another, stabilized by an interfacial film of surfactant. This dispersion may be either oil-in-water or water-in-oil. Microemulsions are typically clear solutions, as the droplet diameter is approximately 100 nanometers or less. The interfacial tension between the two phases is extremely low. Microemulsions are two phase systems.

However it may be desirable to introduce a number of different actives at the same time. In conventional dosage forms hydrophilic and lipoplilic actives were separately prepared to avoid problems relating to their physical stability, such as turbidity, separation and precipitation. This is inconvenient as it requires double handling of each formulation if the desired treatment incorporates hydrophilic and lipophilic actives.

In addition it is desirable to have stable compositions which do not need mixing prior to administration. This is particularly desirable where dealing with administration of a composition to a herd of animals. In general the veterinary formulations are provided in 20 L containers suitable for the mass administration. Liquids in this volume of container are heavy and effective mixing is hard to achieve. If effective mixing is not achieved it is likely effective administration to all herd members will not occur—some animals will receive a low dose of the active and others will receive a high dose of the active.

This is less of a problem for administration of compositions to humans or to pet animals as in general dosages are individually packed in volumes of less than 500 mL. However stable compositions are still desirable as it is sometimes unsuitable to shake up a composition to ensure the active is distributed through the formulation.

In addition hydrophilic actives or solutions of actives in hydrophilic solvents are not miscible or soluble with the oil based carriers. Oil based carriers can be used for injections to achieve longer blood levels to avoid the need for repeated doses and to obtain adequate protection.

There is a need for compositions that allow the incorporation of lipophilic and hydrophilic actives and solutions of actives in hydrophilic solvents into oily vehicles.

Objects

It is an object of this present invention to provide an improved composition, or one which will at least provide the public with a useful choice.

Statement of Invention

In a first aspect the invention relates to a stable composition including at least one hydrophilic active and medium chain mono- and di-glycerides.

Preferably the composition additionally includes at least one lipophilic active.

Preferably the composition additionally includes at least one of a surfactant, solvent and a carrier.

More preferably the carrier is selected from the group including non-toxic oils, their purified derivatives and mixtures thereof.

More preferably the carrier is selected from the group including sesame oil, castor oil, olive oil, soybean oil, corn oil, cottonseed oil, peanut oil, medium chain triglycerides, ethyl oleate, disopropyl myristate, benzyl benzoate, and the like.

More preferably the lipophilic active is selected from the group including macrocyclic lactones, including avermectins and milbemycins.

Preferably the hydrophilic active is selected form the group including clorsulon, closantel, vaccine solution, sodium selenate, vitamin B12, and other hydrophilic anthelmintics and mineral salts.

More preferably the organic solvent is selected from the group including benzyl alcohol, polyethylene glycol (PEG), propylene glycol, alcohol including isopropyl, amyl and benzyl, 2-pyrollidone, N-methyl pyrollidone, tetraglycol, dimethyl sulphoxide, xylene, petroleum distillate, aromatic hydrocarbons, dipropylene glycol methyl ether, diethylene glycol monobutyl ether, dioxolans, glycerol formal, glycofurol, dimethylacetamide, N-(B-hydroxyethyl)-lactamide, ethyl lactate, glycerin, 1,3-Butylene glycol and other pharmaceutically acceptable excipients.

Most preferably said organic solvent may be benzyl alcohol.

Preferably the surfactant is selected from the group non-ionic surfactants including polysorbate 85 and polysorbate 80.

Preferably the components are present in such proportions such that they form microemulsions on addition of water.

Preferably the composition is able to incorporate water to form a homogeneous stable product.

Preferably the water uptake capacity of the composition may be between 0 and 15%

Preferably the compositions of the present invention can be easily adapted to be suitable for oral, nasal, ophthalmic and, topical administration in addition to subcutaneous and intra muscular administration.

In another related aspect the invention relates to a method of preparing a stable composition wherein, the active or actives are solubilised in the medium chain mono- and di-glycerides, optionally organic solvents and a surfactant may be added thereto and the resultant formulation is slowly added to a second solution that includes a carrier and other lipophilic excipients. Optionally water can be added as a final step.

In a further related aspect the invention relates to a method of treating animals by administration to said animals of a composition in accordance with the present invention.

### PREFERRED EMBODIMENT

The present invention will be described by way of examples with reference to the following examples.

It has been discovered the inclusion of medium chain mono- and di-glycerides enables the blending of a hydrophilic active or solvent into a composition with a lipophilic active or an oily carrier. The resultant compositions are self emulsifying and stable.

In addition we have identified certain features which further increase the incorporation of water into the composition of the present invention.

We have found the compositions including medium chain mono- and di-glycerides are able to incorporate up to about 15% water. The water incorporation can be increased further by the use of additional excipients as disclosed herein.

After devising a base formulation we conducted further experiments to determine what other factors affect the uptake of water into the compositions.

### EXAMPLE 1

### Abamectin and B12

The base formulation used for the water uptake trials and the method of making same is set out in table 1 below. The abamectin was selected for the lipophilic active and B12 (hydroxocobalamine acetate) for the hydrophilic active. While Capmul MCM was used as the medium chain mono- and di-glycerides in the following examples, those skilled in the art will appreciate that other mixtures of medium chain mono- and di-glycerides including different ratios of mono- and di-glycerides and other ratios of the medium chain lengths are useable in the invention.

In addition while water incorporation is shown, however it will be appreciated the water can be replaced by any desired additional hydrophilic active, e.g. a solution of vaccine.

TABLE 1
Basic formulation for stable incorporation of
hydrophilic actives or solvents and
lipophilic actives or carriers.

| Code | AA (g) | Step |
|---|---|---|
| Abamectin | 0.0515 | 4 |
| Benzyl alcohol | 0.40 | 5 |
| Sesame oil | 1.30 | 1 |
| Ethyl oleate | 1.00 | 2 |
| Medium chain mono- and di- Glycerides | 1.40 | 3 |
| B12 (hydroxo.) | 0.010 | 7 |
| Polysorbate 80 | 0.30 | 6 |
| Water | 0.25 (5%) | 8 |
| Results (Clear) | Yes | |

Method

The abamectin and B12 are mixed in the benzyl alcohol, to form solution 1. Solution 1 is then warmed in a water bath and mixed by vortex for small volumes or mechanical stirring for large volumes until the abamectin is completely dissolved. Polysorbate 80 is then added into stock solution 1. In another container sesame oil, ethyl oleate and medium chain mono- and di-glycerides are mixed by a vortex for small volumes or by a homogenizer for larger volumes until a clear solution is obtained. It is critical solution 1 be added slowly into the mixture of solution 2. This mixture can then be mixed with a vortex or homogenizer. Once thoroughly mixed the sterile water or if preferred vaccine concentrate can be added. The resulting solution should then be mixed gently. The length of the mixing times required at various steps depends on the size of the batch being prepared. By way of example a batch of 5 litres will typically require a mixing time of about 15 minutes.

Water Incorporation

Water incorporation was investigated by varying amounts of sesame oil, ethyl oleate and medium chain mono- and di-glycerides while maintaining the ratio of the surfactant and solvent, in this case polysorbate 80 and benzyl alcohol in a ratio of 2:15 and 7:15 respectively. In system A, samples contained 3.9 g oil mixture and 0.85 g of the mixture of polysorbate 80 and benzyl alcohol. This total weight of 40.75 g is equivalent to 5 ml.

In system B, the oil mixture of 3.6 g was mixed with 1.1 g mixture of polysorbate 80 and benzyl alcohol. A 4.7 g formulation is equivalent to 5 ml.

All the samples were left overnight at room temperature and at 4° C. before visual observation. Samples were checked for precipitation, turbidity and separation of the surfactants. A clear isotropic region indicates the formation of microemulsions.

Formulations of abamectin, a lipophilic active, and B12 (hydroxocobalamin acetate), a hydrophilic active, were prepared using this technique. From the table below it can be seen an oily solution of abamectin 10 mg/ml and hydroxocobalamin acetate 2 mg/ml can be readily prepared and found to have an improved physical stability.

TABLE 2
Abamectin 10 mg/ml and hydroxocobalamin
acetate 2 mg/ml - Self Emulsifying
Microemulsions For Injection.

| Code | A(g) | B(g) |
|---|---|---|
| Abamectin | 0.0515 | 0.0515 |
| Benzyl alcohol | 0.75 | 0.75 |
| Sesame oil | 1.90 | 2.00 |
| Ethyl oleate | 1.00 | — |
| Medium chain mono- and di-glycerides | 1.00 | 1.66 |
| B12 (hydroxo.) | 0.010 | 0.01 |
| Polysorbate 80 | 0.10 | 0.35 |
| Results (Clear) | Yes | Yes |

TABLE 3
Samples were added with a constant ratio of 2:15 of polysorbate 80:benzyl alcohol.

| | | Medium chain | | | | |
|---|---|---|---|---|---|---|
| | | mono- and di- | Polysorbate 80 | | | |
| Sesame oil | Ethyl oleate | glycerides | and benzyl alcohol | Water | Total | % Water |
| 0 | 0 | 3.9 | 0.85 | 0.65 | 5.40 | 12.0 |
| 1.95 | 0 | 1.95 | 0.85 | 0.25 | 5.00 | 5.0 |
| 0 | 1.95 | 1.95 | 0.85 | 0.25 | 5.00 | 5.0 |
| 1.95 | 0.975 | 0.975 | 0.85 | 0.05 | 4.80 | 1.0 |
| 0.975 | 1.95 | 0.975 | 0.85 | 0.05 | 4.80 | 1.0 |
| 0.975 | 0.975 | 1.95 | 0.85 | 0.25 | 5.00 | 5.0 |
| 0 | 3.12 | 0.78 | 0.85 | 0.05 | 4.80 | 1.0 |
| 1.56 | 1.56 | 0.78 | 0.85 | 0.1 | 4.85 | 2.1 |
| 0 | 3.51 | 0.39 | 0.85 | 0 | 4.75 | 0.0 |
| 0 | 3.71 | 0.19 | 0.85 | 0 | 4.75 | 0.0 |
| 1.17 | 2.34 | 0.39 | 0.85 | 0 | 4.75 | 0.0 |
| 2.73 | 0 | 1.17 | 0.85 | 0 | 4.75 | 0.0 |
| 2.34 | 0.78 | 0.78 | 0.85 | 0 | 4.75 | 0.0 |

TABLE 4
Samples were provided with a constant ratio of 7:15 polysorbate 80:benzyl alcohol.

| | | Medium chain | | | | |
|---|---|---|---|---|---|---|
| | | mono- and di- | Polysorbate 80 | | | |
| Sesame oil | Ethyl oleate | glycerides | and benzyl alcohol | Water | Total | % Water |
| 0 | 0 | 3.6 | 1.1 | 0.85 | 5.55 | 15.3 |
| 1.8 | 0 | 1.8 | 1.1 | 0.15 | 4.85 | 3.1 |
| 0 | 1.8 | 1.8 | 1.1 | 0.3 | 5.00 | 6.0 |
| 1.8 | 0.9 | 0.9 | 1.1 | 0 | 4.70 | 0.0 |
| 0.9 | 1.8 | 0.9 | 1.1 | 0.2 | 4.90 | 4.1 |
| 0.9 | 0.9 | 1.8 | 1.1 | 0.35 | 5.05 | 6.9 |
| 0 | 3.24 | 0.36 | 1.1 | 0 | 4.70 | 0.0 |
| 2.16 | 0 | 1.44 | 1.1 | 0 | 4.70 | 0.0 |
| 1.28 | 1.8 | 0.6 | 1.1 | 0 | 4.78 | 0.0 |
| 0.72 | 2.52 | 0.36 | 1.1 | 0 | 4.70 | 0.0 |

The majority of the samples were clear when the water was added to the systems. The water uptake capacity is in a range of 0% to 15%. As can be seen from the results above water uptake capacity increases with increasing amounts of medium chain mono- and di-glycerides.

The above results provide the basis of the system in which hydrophilic and lipophilic actives can be combined in a stable formulation in a predictable way. The example below shows an appropriate composition in which abamectin, hydroxocobalamin acetate and water up to 5% stably coexist. The composition forms a self emulsifying microemulsion and demonstrated good physical stability during storage at room temperature and also at 4° C. The water in the solution could be replaced by an aqueous solution such as a concentrated vaccine.

The potential use of this system is to incorporate additives in the form of an aqueous solution such as a vaccine concentrate, together with a hydrophilic and lipophilic drug. An appropriate formula can solubilize abamectin, hydroxocobalamin acetate and water up to 5% (see table below). Self emulsifying microemulsions containing abamectin, hydroxocobalamin acetate and water demonstrated a good physical stability during storage at room temperature and 4° C.

Generally we have found the main factors affecting the incorporation of water into the composition are:

The solvent listed in the specification above affect the incorporation of water.

Suitable ratios of oils (e.g. sesame oil and ethyl oleate) increases the incorporation of water.

Medium chain mono- and di-glycerides and polysorbate 80 increases the incorporation of water.

Using the above system described above we have identified the following preferred compositions incorporating lipophilic and hydrophilic actives:

EXAMPLE 2
Macrocyclic Lactone And Closantel Combination

| | |
|---|---|
| Abamectin | 1% |
| Closantel Sodium | 10% |
| Benzyl Alcohol | 15% |
| Sesame Oil | 17% |
| Ethyl Oleate | 27% |
| medium chain mono- | 25% |
| and di-glycerides | |
| Polysorbate 80 | 2% |
| PEG 400 | 3% |

EXAMPLE 3
Macrocyclic Lactone And Selenium Combination

| | |
|---|---|
| Abamectin | 1% |
| Sodium Selenate | 0.3% |
| Benzyl Alcohol | 6% |
| Sesame Oil | 6% |
| Ethyl Oleate | 34% |
| medium chain mono- | 31.7% |
| and di-glycerides | |
| Polysorbate 85 | 3% |
| PEG 400 | 11% |
| Distilled water | 7% |

EXAMPLE 4
Macrocyclic Lactone and clorsulon combination

| | |
|---|---|
| Avermectin | 1% |
| Clorsulon | 10% |
| Benzyl Alcohol | 16% |
| Sesame Oil | 5% |
| Ethyl Oleate | 34% |
| medium chain mono- | 25% |
| and di-glycerides | |
| Polysorbate 85 | 3% |
| PEG 400 | 6% |

EXAMPLE 5
Macrocyclic Lactone and Clorsulon Combination

| | |
|---|---|
| Ivermectin | 1% |
| Clorsulon | 10% |
| Benzyl Alcohol | 10% |
| Capmul PG8 | 10% |
| PEG400 | 30% |
| Glycerol | 19% |
| medium chain mono- | 20% |
| and di-glycerides | |

Throughout the description of this specification the word “comprise” and variations of that word, such as “comprises” and “comprising”, are not intended to exclude other additives, components, integers or steps.

Advantages

The development of a system that is capable stably incorporating hydrophilic actives into formulations including lipophilic or oily compositions is advantageous.

With respect to administration of compositions to animals the farmer would previously had to either administer hydrophilic and lipophilic actives, one after the other in separate runs or alternately would have to mix the various formulations for administration together immediately prior to administration to the animal. This immediacy has disadvantages in that it requires the farmer to premix the formulation in the field and the mixing may not be in the right proportions or may not be thorough so the animals do not receive the proper dosage of each active.

With respect to the administration of compositions to humans the doctor will previously have administered the two actives separately. This is inefficient and uneconomic.

In addition providing a composition that stably combines the hydrophilic active in an oily formulation extends the blood life of both the hydrophilic and lipophilic actives.

These systems are physically stable. They form microemulsions spontaneously when mixed with water in certain proportions without using a high input of energy in the manufacturing process. This formulation is therefore practical for the production in the manufacturing scale. Clear isotropically stable microemulsions can ensure the homogeneity of the hydrophilic and lipophilic drugs in the formulation. The separation or precipitation of these drugs would lead to the inadequate treatment or toxicity in animals. In this invention, formulations with low concentration of surfactant are successfully prepared. These formulations can avoid problems regarding irritation from a high concentration of surfactant, which is usually used in the preparation of microemulsions. medium chain mono- and di-glycerides can be used to increase oral absorption and percutaneous absorption of drug. The appropriate formula containing a low concentration of surfactant and solvent can be used for the other forms of administration of hydrophilic and lipophilic drugs. The combination of hydrophilic and lipophilic drugs in the same formula is beneficial in terms of time, costs savings, homogeneous, physically stable formulations and potential use for treatment.

Variations

Finally, various alterations or modifications may be made to the foregoing without departing from the spirit or scope of this invention.

we claim:

1. A stable composition including at least one hydrophilic active and medium chain mono- and di-glycerides.

2. A composition as claimed in any previous claim which additionally includes at least one lipophilic active.

3. A composition as claimed in any previous claim that additionally includes at least one of a surfactant, a solvent and a carrier.

4. A composition as claimed in any previous claim wherein the carrier is selected from the group including non-toxic oils, their purified derivatives and mixtures thereof.

5. A composition as claimed in any previous claim wherein the solvent is selected from the group including, benzyl alcohol, polyethylene glycol (PEG), propylene glycol, alcohol including isopropyl, amyl and benzyl, 2-pyrollidone, N-methyl pyrollidone, tetraglycol, dimethyl sulphoxide, xylene, petroleum distillate, aromatic hydrocarbons, dipropylene glycol methyl ether, diethylene glycol monobutyl ether, dioxolans, glycerol formal, glycofurol, dimethylacetamide, N-(B-hydroxyethyl)-lactamide, ethyl lactate, glycerin, 1,3-Butylene glycol and other pharmaceutically acceptable excipients.

6. A composition as claimed in any previous claim wherein the lipophilic is selected from the group including macrocyclic lactones, including avermectins and milbemycins.

7. A composition as claimed in any previous claim wherein the surfactant is selected from the group including non-ionic surfactants.

8. A composition as claimed in any previous claim wherein the hydrophilic active is selected form the group clorsulon, closantel, vaccine solution, sodium selenate, vitamin B 12, and other hydrophilic anthelmintics and mineral salts.

9. A composition as claimed in any previous claim wherein the surfactant is selected from the group including polysorbate 85 and polysorbate 80.

10. A composition as claimed in any previous claim wherein the components are present in such proportions they form microemulsions.

11. A composition as claimed any previous claim wherein the composition is able to stably incorporate water.

12. A method of preparing a stable composition as claimed in any previous claim wherein the the active or actives are dissolved in an organic solvent and a surfactant is added thereto, the resultant formulation is added slowly to a second solution including a carrier and other lipophilic excipients, optionally water can be added as a final step.

13. A method of treating non-human warm blooded animals by administration to said animals of a composition as claimed in any of claims 1-11.
